(19) United States
(12) Patent Application Publication
(10) Pub. No.: US 2008/0234361 A1
(43) Pub. Date: Sep. 25, 2008

(54) Pharmaceutical Compositions, Methods of Formulation Thereof and Methods of Use Thereof

(75) Inventor: Ephraim Lansky, Haifa (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Rimonest Ltd., Haifa (IL)

(21) Appl. No.: 10/599,146

(22) PCT Filed: Mar. 23, 2005
(86) PCT No.: PCT/IL05/00332

§ 371 Date: Sep. 21, 2006

Related U.S. Application Data
(60) Provisional application No. 60/555,320, filed on Mar. 23, 2004.

(51) Int. Cl. A61K 31/352; A61K 31/202; A61K 31/192; A61P 3/10; A61P 35/04; A61P 3/04

(52) U.S. Cl. 514/453; 514/560; 514/456; 514/570

(57) ABSTRACT

A pharmaceutical composition including a physiologically effective amount of a mixture of conjugated fatty acid(s) and polyphenol(s) is disclosed. Methods of formulation are further disclosed. Trienoic acids such as punicic acid are employed in exceptionally preferred embodiments of the invention. The invention has utility in management/treatment of a wide range of clinical conditions including, but not limited to, obesity, diabetes, cancer and heart disease.

### FIELD AND BACKGROUND OF THE INVENTION

The present invention relates to pharmaceutical compositions, methods of formulation thereof and methods of use thereof and, more particularly, to pharmaceutical compositions based upon a mixture of conjugated fatty acid(s) and polyphenol(s). Utility in management of clinical problems including diabetes, atherosclerosis, cell proliferation disorders, and obesity is disclosed.

Conjugated fatty acids are fatty acids which contain alternating double bonds between adjacent carbon atoms. In between these double bonds occur single bonds. Such fatty acids are known to inhibit prostaglandin biosynthesis, and such can be considered antieicosanoid agents. Conjugated fatty acids are also weakly antioxidant. However, conjugated fatty acids typically occur in nature in complex mixtures such as those present in vegetable oils and animal fats. These mixtures dilute, or mask, the potentially important biological activity of specific conjugated fatty acids.

Polyphenols, including tannins and flavonoids, are complex carbon skeletons to which are affixed at least two hydroxyl groups to different carbon atoms. Such compounds are often potently antioxidant, and may also be estrogenic. Polyphenols are also mild inhibitors of prostaglandin biosynthesis.

Although conjugated fatty acids are potently antieicosanoid, they have as an inherent disadvantage, only weak antioxidant properties. Although polyphenols are potently antioxidant they are, as a group, characterized by weak antieicosanoid activity.

There is thus a widely recognized need for, and it would be highly advantageous to have, pharmaceutical compositions, methods of formulation thereof and methods of use thereof devoid of the above limitation(s).

### SUMMARY OF THE INVENTION

According to one aspect of the present invention there is provided a pharmaceutical composition, the composition including a physiologically effective amount of a mixture of at least one conjugated fatty acid and at least one polyphenol, a pharmaceutically effective carrier and excipients.

According to another aspect of the present invention a mixture of at least one conjugated fatty acid and at least one polyphenol is employed in the manufacture or preparation of a medicament.

According to further features in preferred embodiments of the invention described below, the conjugated fatty acid includes a trienoic acid.

According to still further features in the described preferred embodiments the trienoic acid includes punicic acid.

According to still further features in the described preferred embodiments the polyphenol includes ellagic acid.

According to still further features in the described preferred embodiments the conjugated fatty acid includes punicic acid and the polyphenol includes ellagic acid.

According to still further features in the described preferred embodiments the polyphenol includes at least one member of the group consisting of caffeic acid and is luteolin.

According to still further features in the described preferred embodiments the conjugated fatty acid includes punicic acid.

According to still further features in the described preferred embodiments the conjugated fatty acid is present as punicic acid and accounts for 30 to 35 percent of the mixture and the polyphenols include at least one member of the group consisting of luteolin and caffeic acid and make up the remaining 65 to 70% of the mixture.

According to still further features in the described preferred embodiments the luteolin and the caffeic acid each account for at least 40% of the polyphenols.

According to still further features in the described preferred embodiments the polyphenol includes a phenolic acid.

According to still further features in the described preferred embodiments the phenolic acid includes at least one member selected from the group consisting of quinic acid and caffeic acid.

According to still further features in the described preferred embodiments wherein the polyphenol includes an ellagi-tannin.

According to still further features in the described preferred embodiments the ellagi-tannin includes at least one member selected from the group consisting of ellagic acid and punicalagin.

According to still further features in the described preferred embodiments the polyphenol includes a flavonoid.

According to still further features in the described preferred embodiments the flavonoid includes at least one member selected from the group consisting of quercetin and luteolin.

According to still further features in the described preferred embodiments the physiologically effective amount includes a dosage in the range of 5 to 25 mg/kg of body weight of a subject treated with the composition.

According to still further features in the described preferred embodiments the pharmaceutical composition is supplied in an orally administrable form.

According to still further features in the described preferred embodiments administration of a pharmaceutical composition according to the present invention constitutes a method for management of diabetes mellitus.

According to still further features in the described preferred embodiments the pharmaceutical composition according to the present invention is supplied as an article of manufacture further including packaging material and instructions for use in management of diabetes mellitus.

According to still further features in the described preferred embodiments administration of a pharmaceutical composition according to the present invention constitutes a method for management of atherosclerotic disease.

According to still further features in the described preferred embodiments the no pharmaceutical composition according to the present invention is supplied as an article of manufacture further including packaging material and instructions for use in management of atherosclerotic disease.

According to still former features in the described preferred embodiments administration of a pharmaceutical composition according to the present invention constitutes a method for treatment of a cell proliferation disorder.

According to still further features in the described preferred embodiments the pharmaceutical composition according to the present invention is supplied as an article of manufacture further including packaging material and instructions for use in treatment of a cell proliferation disorder.

According to still further features in the described preferred embodiments the cell proliferation disorder is cancer, more preferably prostate cancer, and practice of the method reduces an invasive capability of cancer cells.

According to still further features in the described preferred embodiments administration of a pharmaceutical composition according to the present invention constitutes a method for treatment of obesity.

According to still further features in the described preferred embodiments the pharmaceutical composition according to the present invention is supplied as an article of manufacture further including packaging material and instructions for use in treatment of obesity.

The present invention successfully addresses the shortcomings of the presently known configurations by providing a pharmaceutical compositions, methods of formulation thereof and methods of use thereof based upon conjugated fatty acid(s) and polyphenol(s). Because oxidative tension can be a trigger for eicosanoid mediated inflammation, a mixture of conjugated fatty acids and polyphenols exhibit a surprising synergy useful in the amelioration of an exceptionally wide range of medical conditions in which both inflammation and oxidative free radical induced cell damage are important mechanisms. Such conditions include, but are not limited to, cancer, cardiovascular disease, type 2 (adult onset) diabetes mellitus and obesity.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a comparative histogram illustrating synergy among active ingredients in pharmaceutical compositions according to the present invention in an in vitro assay of the invasiveness of PC-3 prostate cancer cells.

### DESCRIPTION OF THE PREFERRED EMBODIMENTS

The present invention is of pharmaceutical compositions, methods of formulation thereof and methods of use thereof which can be employed in management or treatment of a wide range of clinical conditions. Specifically, the present invention can be used to treat maldies, including. But not limited to, diabetes, atherosclerosis, cell proliferation disorders, and obesity is disclosed.

Novelty of the present invention lies in the combination of conjugated fatty acid(s) and polyphenol(s). These compounds may be derived from natural sources (e.g. plant or animal extracts) or produced synthetically.

The principles and operation of pharmaceutical compositions, methods of formulation thereof and methods of use thereof according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the conjugated fatty acid and polyphenol mixture accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of is administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be no permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (mixture) effective to prevent, alleviate or ameliorate symptoms of a disorder (e.g., coronary artery occlusion) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress angiogenesis (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

The present invention is primarily embodied by a pharmaceutical composition which includes a physiologically effective amount of a mixture of at least one conjugated fatty acid and at least one polyphenol. The pharmaceutical composition may further include a pharmaceutically effective carrier and/or excipients as detailed hereinabove.

In other words, the present invention lies in mixing at least one conjugated fatty acid and at least one polyphenol in order to manufacture or prepare a medicament.

Preferably, the conjugated fatty acid includes a trienoic acid. Among the trienoic acids, punicic acid has been found to have especial utility in the context of the present invention as demonstrated by examples 1-5 set forth hereinbelow.

With regard to the polyphenolic portion of the pharmaceutical composition, ellagic acid has been found to have especial utility in the context of the present invention as demonstrated by examples 1-5 set forth hereinbelow. This is especially true if the ellagic acid is present in a pharmaceutical composition including punicic acid.

Thus a pharmaceutical composition including a conjugated fatty acid that includes punicic acid and a polyphenol that includes ellagic acid is an especially preferred embodiment of the invention.

Alternately, or additionally, a pharmaceutical composition including a polyphenol that includes caffeic acid and/or luteolin is preferred as demonstrated by examples 1-5 set forth hereinbelow.

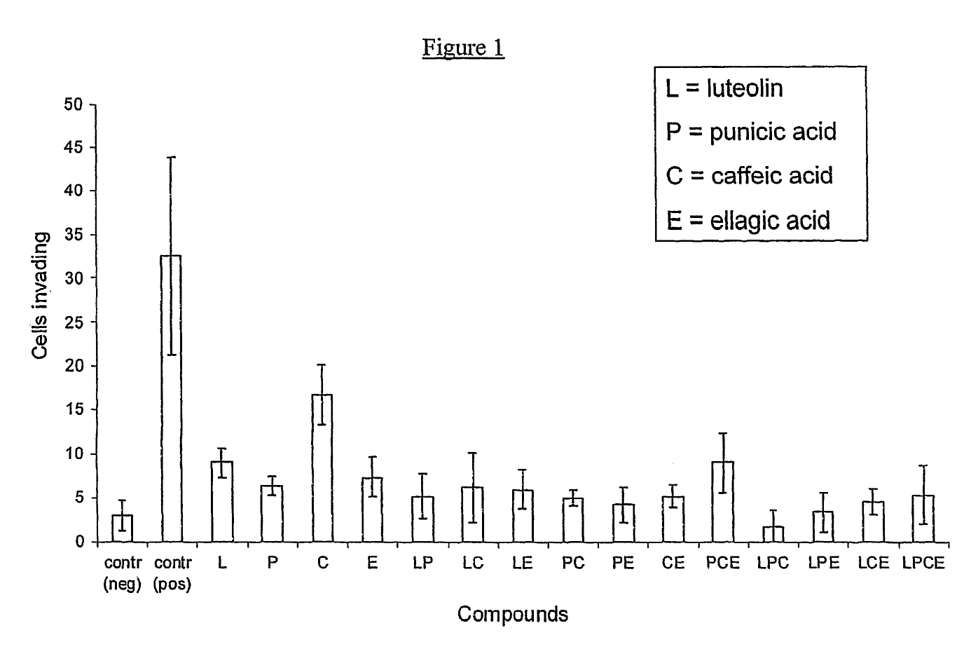

According to a most preferred embodiment of the invention (see Example 6 and FIG. 1) the conjugated fatty acid is present as punicic acid and comprises 30 to 35 percent of the mixture and the polyphenols include luteolin and/or caffeic acid and make up the remaining 65 to 70% of the mixture. Most preferably luteolin and caffeic acid are present in approximately equal amounts.

According to additional preferred embodiments the polyphenol includes a phenolic acid such as, for example, quinic acid and/or caffeic acid as detailed in examples 1-5 hereinbelow.

According to additional preferred embodiments the polyphenol includes an ellagi-tannin such as, for example, ellagic acid and/or punicalagin as detailed in examples 1-5 hereinbelow.

According to additional preferred embodiments the polyphenol includes a flavonoid such as, for example, quercetin and/or luteolin as detailed in examples 1-5 hereinbelow.

It is currently believed that a physiologically effective amount of a mixture according to the present invention is preferably a dosage in the range of 5 to 25 mg/kg of body weight of a subject treated with the composition as set forth in examples 1-5 hereinbelow.

Optionally, but preferably, a pharmaceutical composition according to the present invention is supplied in an orally administrable form (e.g. pill, tablet, capsule, elixir or syrup).

As detailed in Example 2, administration of a pharmaceutical composition according to the present invention may be employed as a method for management of diabetes mellitus, especially type 2 diabetes (i.e. adult onset).

Thus, it may be advantageous to supply a pharmaceutical composition according to the present invention as an article of manufacture further including packaging material and instructions for use in management of diabetes mellitus.

As detailed in Example 3, administration of a pharmaceutical composition according to the present invention may be employed as a method for management of atherosclerotic disease. Thus, it may be advantageous to supply a pharmaceutical composition according to the present invention as an article of manufacture further comprising packaging material and instructions for use in management of atherosclerotic disease. While example 3 relates to treatment of acute clinical symptoms of advanced coronary artery blockage, the present invention can be advantageously employed in prevention of formation of atherosclerotic plaques.

As detailed in Examples 4 and 5, administration of a pharmaceutical composition according to the present invention may be employed as a method for management of a cell proliferation disorder (e.g. cancer).

Thus, it may be advantageous to supply a pharmaceutical composition according to the present invention as an article of manufacture further comprising packaging material and instructions for use in treatment of a cell proliferation disorder. The term treatment as used herein refers to prophylactic as well as palliative as well as curative treatment.

The present invention is especially useful in treatment of cancer, more preferably prostate cancer, and has demonstrable ability to reduce an invasive capability of cancer cells as demonstrated by Example 6.

As detailed in Example 1, administration of a pharmaceutical composition according to the present invention may be employed as a method for treatment of obesity. Further, it is apparent from Example 1 that pharmaceutical compositions according to the present invention have a previously unknown capacity to act as appetite suppressants.

Thus, it may be advantageoust to supply a pharmaceutical composition according to the present invention as an article of manufacture further comprising packaging material and instructions for use in appetite suppression in general and/or treatment of obesity in particular.

Additional objects, advantages, and novel features of the present invention will become apparent to one ordinarily skilled in the art upon examination of the following examples, which are not intended to be limiting. Additionally, each of the various embodiments and aspects of the present invention delineated hereinabove and as claimed in the claims section below finds experimental support in the following examples.

### EXAMPLES

Reference is now made to the following examples, which together with the above descriptions, illustrate the invention in a non limiting fashion.

TABLE 1
First exemplary formulation of a pharmacutical
composition according to the present invention
(only active ingredients listed).

| | | Amount | % total | | |
|---|---|---|---|---|---|
| compound | Type* | (mg) | mixture | % CFA | % pph |
| Punicic acid | cfa | 1000 | | | NA |
| All cfa | | 1000 | 87.33 | 100 | NA |
| punicalagin | pph | 25 | 2.18 | NA | 20.0 |
| luteolin | pph | 20 | 1.748 | NA | 13.8 |
| quercetin | pph | 20 | 1.748 | NA | 13.8 |
| kaempferol | pph | 20 | 1.748 | NA | 13.8 |
| Ellagic acid | pph | 20 | 1.748 | NA | 13.8 |
| Caffeic acid | pph | 20 | 1.748 | NA | 13.8 |
| Quinic acid | pph | 20 | 1.748 | NA | 13.8 |
| All pph | NA | 145 | 12.66 | NA | 100 |

*cfa = conjugated fatty acids; pph = polyphenols

TABLE 2
Second exemplary formulation of a pharmacutical
composition according to the present invention
(only active ingredients listed).

| | | | | | |
|---|---|---|---|---|---|
| | | Amount | % total | | |
| compound | Type* | (mg) | mixture | % CFA | % pph |

| | | | | | |
|---|---|---|---|---|---|
| Punicic acid | cfa | 500 | | 100 | NA |
| All cfa | | 500 | 83.33 | 100 | NA |
| punicalagin | pph | 20 | 3.33 | NA | 20 |
| luteolin | pph | 20 | 3.33 | NA | 20 |
| quercetin | pph | 20 | 3.33 | NA | 20 |
| Ellagic acid | pph | 20 | 3.33 | NA | 20 |
| Caffeic acid | pph | 20 | 3.33 | NA | 20 |
| All pph | NA | 100 | 16.67% | NA | 100 |

*cfa = conjugated fatty acids; pph = polyphenol

### Example 1

### Conjugated Fatty Acid and Polyphenol Mixture in the Treatment of Obesity

A forty year old, 155 cm, 75 kg female subject with gradually increasing obesity for 1 year was treated with a mixture according to the present invention as detailed in table 1 hereinabove. The subject consumed a single 1145 mg. oral dose per day. Improvement was reported by the patient after one week. Improvement manifested as enhanced well-being and a decreased appetite, especially for sweets. These changes persisted throughout one month of treatment, during which the subject decreased her body weight by 5 Kg. The subject remained on the treatment regimen for the next three months and lost an additional 10 kg and reported enhanced well-being and a more easily controlled appetite.

While this example is anecdotal and the well-being and appetite variables are subjective, the objectively measured 15 Kg weight loss over a period of months indicate that pharmaceutical compositions according to the present invention have great potential in controlling obesity.

### Example 2

### Conjugated Fatty Aced and Polyphenol Mixture in the Management of Diabetes

A forty seven year old, 60 kg male subject with adult onset diabetes of three years had been maintained on conventional oral anti-hypoglycemic medicine. Using the aforementioned conventional methods, the subject maintained blood sugar levels in the range of 170±10 according to a patient operated glucose meter employed once every two days. The subject began taking a mixture according to the present invention as detailed in table 1 hereinabove twice daily (i.e. two 1145 mg oral doses per day) as a supplement to the aforementioned conventional oral anti-hypoglycemic medicine. After 10 days, the patient reported a consistent decrease in his average blood sugar levels to 140±10. After two months of regularly taking the mixture according to the present invention (in addition to the conventional oral anti-hypoglycemic medicine), his blood sugar levels remained constant at 140±10. There were no other changes to his daily oral regimen or diet.

While this example is anecdotal the objectively measured decrease in blood glucose levels over a period of months indicate that pharmaceutical compositions according to the present invention have great potential in controlling blood glucose in diabetic patients.

### Example 3

### Conjugated Fatty Acid and Polyphenol Mixture in the Treatment of Atherosclerotic Placques

A seventy two year old male subject presented with mild, but gradually worsening intermittent abulia and bilateral upper extremity numbness and weakness. Carotid artery examination by ultra-sound revealed 90% left sided occlusion and 70% right sided occlusion. A carotid surgical intervention was scheduled thirty days in the future. During the intervening thirty days, the subject began taking a mixture according to the present invention as detailed in Table 1 hereinabove once daily (i.e. one 1145 mg oral dose per day). After thirty days, the subject reported that all symptoms had disappeared. A follow-up examination by ultrasound showed an improvement in his carotid occlusive status to 60% left sided occlusion and 40% right sided occlusion. The attending physician decided to cancel the surgical procedure based upon this documented improvement. The patient has continued on the daily regimen of conjugated fatty acids and polyphenols for four months without recurrence of symptoms.

While this example is anecdotal, the objectively measured improvement in the level of occlusion of major coronary arteries, and the stabilization of the clinical picture over a period of months, suggests that pharmaceutical compositions according to the present invention have great potential in prevention and/or treatment of atherosclerosis.

### Example 4

### Conjugated Fatty Acid and Polyphenol Mixture as an Adjunct Therapy in Cancer Patients

A sixty two year old female subject that had previously undergone bilateral oophorectomy and was currently diagnosed with left sided ductal breast cancer presented with the following serum tumor marker levels:

| | | | |
|---|---|---|---|
| CA-125 | 17.76 | U/ml | range 0-35 |
| CA-15-3 | 25 | IU/L | range 0-31 |
| CEA | 1-30 | ng/ml | range 1-3. |

A left mastectomy was performed, and the subject began treatment with a mixture according to the present invention as detailed in Table 1 (i.e. one 1145 mg oral dose per day). After six months on this regimen, the subject remained free of cancer in the contralateral (i.e. right) breast and there was no elevation in any of the serum markers. The subject also tolerated the mixture without difficulty.

While this example is anecdotal, the objectively measured serum tumor markers suggest that pharmaceutical compositions according to the present invention have great potential as an adjunct therapy in metastatic disease.

### Example 5

### Conjugated Fatty Acid and Polyphenol Mixture in the Treatment of Cancer

A female subject was diagnosed with histiocytoma at age 50 and underwent surgery to remove an orange-sized abdominal tumor. At age 56 a recurrence was noted and again an orange-sized tumor was surgically removed. Two months after surgery the subject presented with abdominal vain and weakness. Computerized abdominal tomography (CT) indicated fatty liver. The subject began treatment with a mixture according to the present invention as detailed in Table 2 hereinabove (i.e one oral dose of 600 mg/day). After one year of this regimen the subject reported a marked reduction in pain and weakness and a CT revealed a complete resolution of the previously observed fatty liver.

While this example is anecdotal and the pain and weakness variables are subjective, the objectively measured elimination of fatty liver suggests that pharmaceutical compositions according to the present invention have great potential in controlling cancer related symptoms and/or preventing recurrence of metastatic disease.

### Example 6

### Conjugated Fatty Acid and Polyphenol Suppress PC-3 Cell Invasion in an In Vitro Assay

In order to determine a possible molecular mechanism of mixtures of conjugated fatty acids and polyphenols according to the present invention, an accepted in vitro assay of cancer cell invasion was employed (Jiang et al., (1995) Regulation of the expression of E-cadherin on human cancer cells by gamma-linolenic acid (GLA). Cancer Res. 55:5043-5048).

Specifically, human PC-3 prostate cancer cells were used as an in vitro model to assess invasion across a Matrigel™ (Becton, Dickenson and Company, Franklin Lakes, N.J.) artificial membrane. Briefly, 6.5 mm diameter polycarbonate membranes (pore size 8 microns) in the chambers of a 24-transwell system (Corning Costar Transwell, Cambridge, Mass.) at 50 micrograms/membrane. Following gel rehydration, 5×10⁴ PC-3 human prostate cancer cells were added to each well. Hepatocyte growth factor/scatter factor (HGF) (Becton, Dickenson & Company, Franklin Lakes, N.J.) was used at 40 ng/ml to induce invasion. The upper chamber contained thested components (i.e. punicic acid, luteolin, caffeic acid or ellagic acid, or a combination of two, three or all four of the chemicals) at a fixed total concentration of 4 micrograms/ml. After 72 hr culture, invasive cells stuck to the lower surface were fixed and stained with crystal violet, and their number quantified under an inverted microscope and expressed as percentage of positive control.

Pure compounds (ellagic acid (B, luteolin [L], caffeic acid [C] and punicic acid [V]) were purchased commercially (E, C and L from Sigma Aldrich, Rehovot, Israel; P from Larodan Chemicals; Malmo; Sweden) and assayed as potential invasion inhibitors. The total dose was 4 micrograms/ml in each of 18 trials. The Kruskal-Wallis test was used to measure significance.

All four compounds individually inhibited invasion significantly, with the effect of caffeic acid much less than that of ellagic acid, punicic acid or luteolin. Combinations of two compounds suggested a trend towards synergy, but the results were not significant. However, combinations with three compounds did result in statistically significant benefits, with the strongest effect from the equal combination of luteolin, punicic acid and caffeic acid. There was no added benefit from combining all four compounds. Results are summarized graphically in FIG. 1 and numerically in table 3.

TABLE 3
Effects of pure chemicals on PC-3 Invasion in an in vitro assay

| Singlets | | Doublets | | Triplets | | Quadruplet | |
|---|---|---|---|---|---|---|---|
| | X ± σ | | X ± σ | | X ± σ | | X ± σ |
| L | 9 ± 1.6 | LP | 5.2 ± 2.6 | PCE | 9.0 ± 3.4 | LPCE | 5.4 + |
| | | | | | | | 3.4 |
| P | 6.4 ± 1.1 | LC | 6.2 ± 4.0 | LPC | 1.8 ± 1.8 | | |
| C | 16.8 ± 3.4 | LE | 6.0 ± 2.2 | LPE | 3.4 ± 2.3 | | |
| E | 7.4 ± 2.3 | PC | 5.0 ± 1.0 | | | | |
| c+ | 33 ± 10.7 | PE | 4.2 ± 2.0 | | | | |
| c− | 3 ± 1.7 | CE | 5.2 ± 1.3 | | | | |

L = luteolin,
P = punicic acid,
C = caffeic acid,
E = ellagic acid,
c+ = positive control.
c− = negative control.
X = mean number of invading cells,
σ = standard deviation

These results demonstrate that purified conjugated fatty acids and polyphenols exhibit a surprising synergy with respect to the invasive capacity of cancer cells and suggest a molecular basis for the clinical observations of examples 4 and 5. In addition, they indicate that combination of conjugated fatty acids, especially trienoic acids, especially punicic acid with one or more polyphenols has tremendous utility a variety of clinical applications.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-27. (canceled)

28. A pharmaceutical composition, the composition comprising a physiologically effective amount of a mixture of at least one conjugated fatty acid and at least one polyphenol, a pharmaceutically effective carrier and excipients.

29. The pharmaceutical composition of claim 28, wherein said conjugated fatty acid includes a trienoic acid.

30. The pharmaceutical composition of claim 29, wherein said trienoic acid includes punicic acid.

31. The pharmaceutical composition of claim 28, wherein said polyphenol includes ellagic acid.

32. The pharmaceutical composition of claim 28, wherein said conjugated fatty acid includes punicic acid and said polyphenol includes ellagic acid.

33. The pharmaceutical composition of claim 28, wherein said polyphenol includes at least one member of the group consisting of caffeic acid and luteolin.

34. The pharmaceutical composition of claim 31, wherein said conjugated fatty acid includes punicic acid.

35. The pharmaceutical composition of claim 28, wherein said conjugated fatty acid is present as punicic acid and comprises 30 to 35 percent of said mixture and said polyphenols include at least one member of the group consisting of luteolin and caffeic acid and comprise the remaining 65 to 70% of said mixture.

36. The pharmaceutical composition of claim 33, wherein said luteolin and said caffeic acid each comprise at least 40% of said polyphenols.

37. The pharmaceutical composition of claim 28, wherein said polyphenol includes a phenolic acid.

38. The pharmaceutical composition of claim 35, wherein said phenolic acid includes at least one member selected from the group consisting of quinic acid and caffeic acid.

39. The pharmaceutical composition of claim 28, wherein said polyphenol includes an ellagi-tannin.

40. The pharmaceutical composition of claim 37, wherein said ellagi-tannin includes at least one member selected from the group consisting of ellagic acid and punicalagin.

41. The pharmaceutical composition of claim 28, wherein said polyphenol includes a flavonoid (e.g., quercetin, luteolin).

42. The pharmaceutical composition of claim 28, wherein said flavonoid includes at least one member selected from the group consisting of quercetin and luteolin.

43. The pharmaceutical composition of claim 28, wherein said physiologically effective amount includes dosages in the range of 5 to 25 mg/kg of body weight of a subject treated with the composition.

44. The pharmaceutical composition of claim 28, supplied in an orally administrable form.

45. A method for management of diabetes mellitus, the method comprising administering a pharmaceutical composition according to claim 28.

46. A pharmaceutical composition according to claim 28 supplied as an article of manufacture further comprising packaging material and instructions for use in management of diabetes mellitus.

47. A method for management of atherosclerotic disease, the method comprising administering a pharmaceutical composition according to claim 28.

48. A pharmaceutical composition according to claim 28 supplied as an article of manufacture further comprising packaging material and instructions for use in management of atherosclerotic disease.

49. A method for treatment of a cell proliferation disorder, the method comprising administering a pharmaceutical composition according to claim 28.

50. A pharmaceutical composition according to claim 28 supplied as an article of manufacture further comprising packaging material and instructions for use in treatment of a cell proliferation disorder.

51. The method of claim 28, wherein said cell proliferation disorder is prostate cancer and practice of the method reduces an invasive capability of said prostate cancer.

52. A method for treatment of obesity, the method comprising administering a pharmaceutical composition according to claim 28.

53. A pharmaceutical composition according to claim 28 supplied as an article of manufacture further comprising packaging material and instructions for use in treatment of obesity.

54. Use of a mixture of at least one conjugated fatty acid and at least one polyphenol in the manufacture or preparation of a medicament.
